(19) United States
(12) Patent Application Publication
(10) Pub. No.: US 2002/0045886 A1
(43) Pub. Date: Apr. 18, 2002

(54) URETHRAL CATHETER

(76) Inventor: CHRISTOPHER H. PORTER, WOODINVILLE, WA (US)

Correspondence Address: PATRICIA A. SHEEHAN, CESARI AND MCKENNA, LLP, 88 BLACK FALCON AVENUE, BOSTON, MA, 02210, US

(21) Appl. No.: 09/290,988

(22) Filed: Apr. 13, 1999

(51) Int. Cl. A61M 25/00; A61M 27/00

(52) U.S. Cl. 604/544; 604/104

(57) ABSTRACT

A urethral catheter includes an upper section that directs urine in a path from the bladder through a top portion of the urethra, a mid-section that includes the walls of the urethra in a path through the mid-portion of the urethra, and a lower section that directs the urine in a path through the lower portion of the urethra to a collection device. As urine flows through the mid-section of the catheter, the urine flows along the portions of the walls of the urethra that are included in the path, and thereby cleanses those portions of bacteria. The mid-section may consist of relatively widely-spaced rods, a metal mesh, or any other construction that is sufficiently rigid to maintain the shape of the path through the mid-portion of the urethra and includes spaces or gaps that include the walls of the urethra in the path provided by the mid-section.

### FIELD OF INVENTION

This invention relates generally to urethral catheters.

### BACKGROUND OF THE INVENTION

Urethral catheters are used to direct urine flow from the bladder to a collection device that is outside of the body. Urethral catheters are used, for example, by post-operative patients who must have their urine collected and/or measured, or other bed-ridden patients. A common type of urethral catheter is a Foley catheter.

A urethral catheter is inserted through the urethra and into the bladder. The catheter thus provides a path for bacteria to travel from the meatus and the distal end of the urethra to the proximal end of the urethra and the bladder. Further, the insertion of the catheter into the urethra often irritates the lining of the urethra, and thus, promotes bacterial growth.

U.S. Pat. Nos. 3,769,981 and 4,878,901 discuss urethral catheters that attempt to combat bacterial migration with perforated or reduced-diameter mid-sections. Theoretically, urine flowing from the bladder through the upper section of the catheter exits the catheter through the perforated or reduced-diameter mid-section, cleanses the section of topical bacteria, to prevent bacterial migration to the upper end of the catheter and into the bladder. The urine then flows into the lower section of the catheter, which directs the urine to the collection device. In practice, the reduced-diameter section of the catheter may not maintain its shape as the catheter is removed, resulting essentially in a folding of the mid-section that may damage the walls of the urethra. The catheter with the perforated mid-section does not necessarily direct urine flow through the perforations. Indeed, most of the urine will flow instead through the center of the catheter along the path of least resistance to the flow. This may also occur with the reduced-diameter catheter. Accordingly, there may not be sufficient urine flow outside of the catheters to remove the topical bacteria. Further, the perforations may weaken the mid-section which may then fold or rip during the removal of the catheter.

### SUMMARY OF THE INVENTION

An improved urethral catheter includes an upper section that directs urine from the bladder through a top portion of the urethra, a mid-section that consists of a plurality of widely-spaced rods, and a lower section that directs urine to a collection device that is positioned outside the body. As urine flows through the mid-section of the catheter, the urine flows along the portions of the walls of the urethra that correspond to the gaps between the rods. The urine then continues to flow through the lower section of the catheter. As needed, the proximal end of the lower section of the catheter may include a lip that is shaped to direct the urine flow through the lower portion of the catheter, and thus, prevent leakage around the catheter.

The metal rods are preferably made of stainless steel, so that bacteria will not adhere to them. The mid-section thus interrupts the path of bacterial migration along the catheter.

Alternatively, the mid-section of the catheter may consist of a metal braid that while stiff enough to retain its shape inside the urethra, is woven relatively loosely such that urine flowing through the braid flows essentially along the walls of the urethra.

The improved urethral catheter has a smooth transition between the various sections, and maintains an essentially uniform diameter along its length. This prevents the catheter from folding at its mid-section when the catheter is removed. Further, urine flowing along the path through the catheter necessarily flows along the walls of the urethra because the urethral walls fill in the gaps between the rods or braided threads.

### BRIEF DESCRIPTION OF THE DRAWINGS

FIG. 1 depicts a urethral catheter constructed in accordance with the invention;

FIG. 2 depicts the urethral catheter of FIG. 1 with an additional lip for directing urine flow through a lower portion of the catheter; and

FIG. 3 depicts an alternative embodiment of the urethral catheter.

### DETAILED DESCRIPTION OF AN ILLUSTRATIVE EMBODIMENT

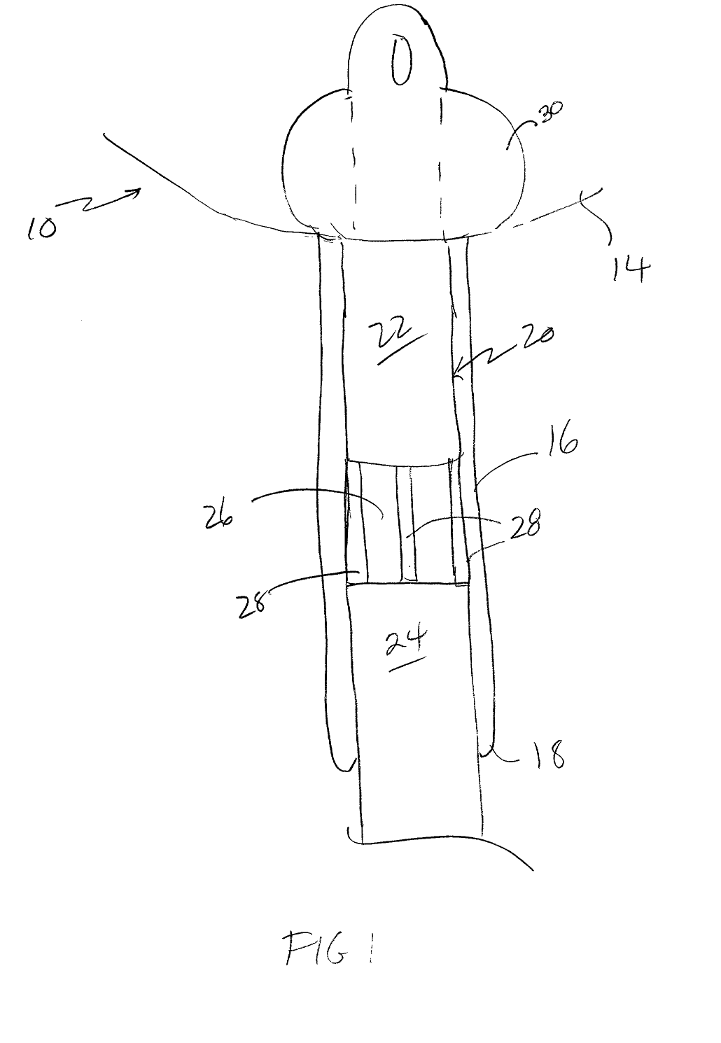

Referring now to FIG. 1, a urethral catheter 20 includes a top section 22 that directs urine flow from a bladder 14 through a top portion of the urethra 16. A lower section 24 of the catheter directs urine from the lower end of the urethra to a collection device (not shown) that is positioned outside of the body 10. The catheter 20 also includes a mid-section 26 that connects the upper and lower sections 22 and 24 of the catheter.

The mid section 26 consists of a plurality of spaced rods 28. The rods are essentially equally spaced around the circumference of the catheter, and there are a sufficient number of rods to ensure that the mid-section will not fold as the catheter is removed. The rods are preferably made from a material to which bacteria do not adhere, for example, stainless steel. The rods may also be coated in silicone to promote removal of the catheter.

When urine flows through the mid-section 26 of the catheter, the urine flows along the walls of the urethra which, because of the reflexive closure of the walls, essentially fill-in the gaps between the rods 28. The flowing urine thus cleanses the portions of the walls with which it comes in contact, and in a natural manner, minimizes the migration of bacteria from the lower section of the urethra and the meatus 18 to the upper section of the urethra and the bladder 14. Further, the path for bacterial migration along the catheter is interrupted because the bacteria do not adhere to the rods 28.

The urethral catheter 20 may include at its upper end a conventional Foley-type opening 21 through which urine flows from the bladder. Also, in a conventional manner, the catheter may include an anchoring mechanism 30, such as a balloon that is inflated after the catheter is inserted and deflated for removal of the catheter.

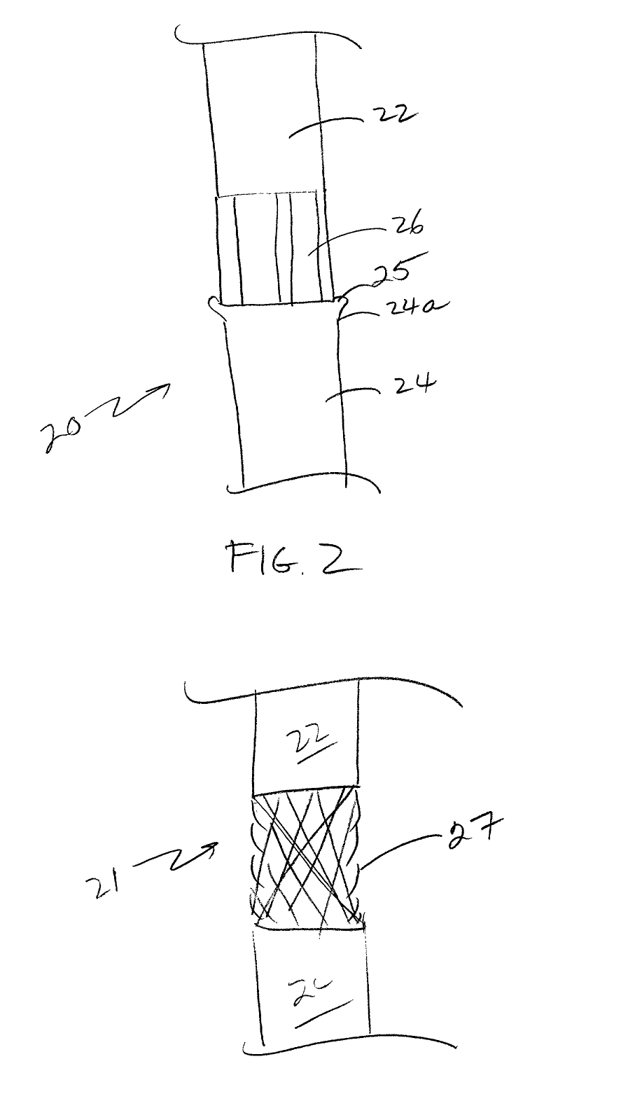

FIG. 2 depicts the urethral catheter 20 with a lip 25 at the top end 24a of the bottom section 24. The lip 25 catches any urine that may otherwise leak past the opening of the lower section of the catheter. The lip 25 has a slightly wider diameter than the lower section, and thus, tends to direct the urine into the lower section of the catheter.

Referring now to FIG. 3, a urethral catheter 21 includes the upper and lower sections 22 and 24 discussed above with reference to FIG. 1, and a mid-section 27 that consists of a metal braid. The metal braid is loosely woven, such that urine flowing through the mid-section of the catheter comes in contact with the urethral walls. The braid, however, is structurally rigid, such that it maintains its shape within the urethra and does not fold or bend in response to the reflexive closure of the walls. As discussed with reference to FIG. 1, urine flowing through the upper section 22 of the catheter flows along the mid-section 27, and thus, along the urethral walls. The urine then flows through the bottom section of the catheter to the collection device, as discussed above.

Each catheter 20 and 21 depicted in FIGS. 1-3 has an essentially uniform outer diameter along its length, and is structurally sound. The catheters thus maintain their shape when they are removed, and do not tend to fold at their mid-sections.

What is claimed is:

1. A urethral catheter for directing urine flow through a urethra, the urethral catheter including:
A. a proximal section for providing a first end of a path for urine flow from a bladder through the urethra;
B. a distal section for providing a second end of the path; and
C. a mid-section between said proximal and distal sections, the mid-section including connecting means for connecting said proximal and distal sections and providing a mid-portion of the path, the connecting mean directing urine flow from the first end of the path, along walls of the urethra in the mid-section of the path to the second end of the path.

2. The urethral catheter of claim 1, wherein the connecting means includes a plurality of rods that are separated from one another to include the walls of the urethra in the mid-section of the path.

3. The urethral catheter of claim 2 wherein the plurality of rods are spaced around a circumference of a distal end of the proximal section and a proximal end of the distal section.

4. The urethral catheter of claim 3, wherein the rods maintain a uniform diameter from the distal end of the proximal section to the proximal end of the distal section.

5. The urethral catheter of claim 4, wherein the rods are sufficiently stiff to maintain the mid-section of the path in a predetermined shape.

6. The urethral catheter of claim 1, wherein a proximal end of the distal section includes a lip portion for directing urine flow from a distal end of the mid-section of the path to a proximal end of the second end of the path.

7. The urethral catheter of claim 1, wherein the connecting means is constructed of a material to which bacteria do not adhere.

8. The urethral catheter of claim 7, wherein the material is stainless steel.

9. The urethral catheter of claim 1, wherein the connecting means includes a woven mesh with gaps through which the walls of the urethra are included in the mid-section of the path.

10. The urethral catheter of claim 9, wherein the mesh maintains a uniform diameter from a distal end of the proximal section to a proximal end of the distal section.

11. The urethral catheter of claim 9, wherein the mesh is sufficiently stiff to maintain the mid-section of the path in a desired shape.

12. The urethral catheter of claim 9, wherein a proximal end of the distal section includes a lip portion for directing urine flow from a distal end of the mid-section of the path to a proximal end of the second end of the path.

13. The urethral catheter of claim 9, wherein the connecting means is constructed of a material to which bacteria do not adhere.

14. The urethral catheter of claim 13, wherein the material is stainless steel.

15. The urethral catheter of claim 8, wherein the mesh comprises a metal braid.

16. A urethral catheter for directing urine flow through a urethra, the urethral catheter including:
A. a proximal section for providing a first end of a path for urine flow from a bladder through the urethra;
B. a distal section for providing a second end of the path; and
C. a mid-section between said proximal and distal sections, the mid-section including connecting means for connecting said proximal and distal sections and providing a mid-portion of the path, the connecting means including as part of the mid-section of the path portions of wall of the urethra.

17. The urethral catheter of claim 16, wherein the connecting means includes a plurality of spaced rods that include in the mid-section of the path the portions of the walls of the urethra that correspond to the spacing between adjacent rods.

18. The urethral catheter of claim 17 wherein the plurality of rods are spaced around a circumference of the mid-section to include in the mid-section of the path portions of all of the walls of the urethra.

19. The urethral catheter of claim 16, wherein the connecting means includes a woven mesh with a plurality of gaps for including in the mid-section of the path the portions of the walls of the urethra that correspond to the gaps.

20. The urethral catheter of claim 19 wherein the gaps are spaced around a circumference of the mid-section to include in the mid-section of the path portions of all of the walls of the urethra.
